(19) United States
(12) Patent Application Publication
(10) Pub. No.: US 2021/0177924 A1
(43) Pub. Date: Jun. 17, 2021

(54) CANNABIS AND BUTTERFLY PEA EXTRACT

(71) Applicant: WICC INVESTMENTS, LLC DBA CORE GARDENS, Maupin, OR (US)

(72) Inventor: RACHEL RYKAL, MAUPIN, OR (US)

(21) Appl. No.: 17/118,854

(22) Filed: Dec. 11, 2020

Related U.S. Application Data
(60) Provisional application No. 62/946,820, filed on Dec. 11, 2019.

(51) Int. Cl. A61K 36/48; A61K 31/05; A61K 31/352; A61K 9/00; A61K 9/20; A61K 9/14; A61K 9/48

(52) U.S. Cl. CPC A61K 36/48; A61K 31/05; A61K 31/352; A61K 9/0014; A61K 9/0056; A61K 9/0095; A61K 9/14; A61K 9/48; A61K 9/20

(57) ABSTRACT

A formulation includes butterfly pea extract and cannabinoids.

### CROSS REFERENCE TO RELATED APPLICATION

This application claims priority to and the benefit of U.S. Provisional Application No. 62/946,820 filed Dec. 11, 2019, which is incorporated hereby reference in its entirety.

### BACKGROUND

The health benefits of various cannabinoids have just begun to receive attention. A “cannabinoid” comprises any of a group of chemical compounds that act on the endocannabinoid system found in cells that alter neurotransmitter release in the brain. Their unique properties and interaction with the neurotransmitters has made them attractive options for pain relief, sleep aids and other physiological uses.

These compounds include tetrahydrocannabinol acid (THCA), tetrahydrocannabivarin (THCV), and tetrahydrocannabinol (THC), the primary psychoactive compounds in cannabis, and the most stringently regulated. Many cannabinoids have their main form, and an acid form, designated by the addition of the letter “A.” The term “THC” as used here encompasses THC, delta-8 and delta-9 THC, THCV and THCA.

Other cannabinoids include cannabidiol/cannabidiolic acid, CBD/CBDA, cannabinol (CBN), cannabigerol (CBG), cannabichromene (CBC), and cannabidivarin (CBDV), without limitation. Some scientists estimate that more than eighty unique cannabinoids exist. Cannabinoids, other than THC compounds, may result from industrial hemp plants, defined as plants having less than 0.3% THC. This renders products made from industrial hemp legally saleable in interstate commerce, or federally.

New formulations of various cannabinoids, probably most commonly CBD at this time, mixed with other types of materials can enhance or increase the effects of the cannabinoids and the compound on the body.

### DETAILED DESCRIPTION

Many different types of plant-based extracts can have beneficial effects on the body such as reducing inflammation and/or pain, as sleep aids, digestive aids, etc. One such plant is the Butterfly Pea Flower, the clitoria ternatea. This flower goes by several different colloquial names, include Asian Pigeon Wings, Blue Bell Vine, Cordofan Pea, and Darwin Pea, but all refer to the clitoria ternatea. Butterfly Pea Tea has been used for centuries according to some sources as a memory enhancer, brain booster, pain reducer and overall calming agent. It contains flavonoids, antioxidants and peptides, among other beneficial properties.

As used here, the term butterfly pea extract (BPE) comprises any substance that results from the butterfly pea flower or plant. While typically in liquid or powder form, including oils, no limitation to that form is intended and none should be implied.

The term “cannabinoid” as used here comprises any of a group of chemical compounds that act on the endocannabinoid system found in cells that alter neurotransmitter release in the brain. Similarly, the term THC, for ease of discussion, will refer to tetrahydrocannabinol acid (THCA), tetrahydrocannabivarin (THCV) and tetrahydrocannabinol (THC), delta-9 THC and delta-8 THC. The term “cannabinoids” as used here includes cannabidiol/cannabidiolic acid (CBD/CBDA), cannabinol (CBN), cannabigerol (CBG), cannabichromene (CBC), and cannabidivarin (CBDV), and the various forms of THC.

The terms “hemp-derived” and “hemp-sourced” refers to the source of cannabinoids as comprising plants that have 0.3% or less THC and qualify under the 2014/2018 Farm Bills as industrial hemp.

By combining cannabinoids and butterfly pea extracts, the two compounds have a beneficial effect on the body and enhance each other's properties. The combination may include hemp-sourced cannabinoids, and/or cannabinoids from plants containing high levels (than 0.3%) of THC, depending upon the regulations and the intended market. No limitation to either source of cannabinoids is intended and none should be implied.

One embodiment comprises a combination of cannabinoids and butterfly pea extract (BPE), in any concentration of cannabinoids and any concentration of BPE.

An embodiment comprises a combination of hemp-derived cannabinoids and BPE.

An embodiment comprises a combination of BPE and cannabinoids derived from plants containing higher levels of THC.

The above embodiments may include other cannabinoids, and those may be hemp-derived or derived from plants having higher levels of THC.

Any of the combinations and formulations listed above or below may take the form of consumables or topicals. Consumables include any item intended for consumption, such as, but not limited to, tinctures, oils, water-soluble tablets or powder packets, capsules, pills, teas, powders, edible items such as gummies and chocolates, and liquids as examples. Topicals include any item spread over the skin and/or hair, such as lotions, balms, oils, shampoos, etc. These may be referred to as topical solutions.

In any of the formulations, the units will generally be in terms of milligrams per ounce, where the ounce is a liquid measure equal to approximately 30 milliliters. Each formulation will have a range of BPE, and total cannabinoids. For consumables, or formulation measures that are an ounce or less, the units will be milligrams, not milligrams per ounce. This includes tinctures. Tinctures are a liquid consumable.

The term “total cannabinoids” as used here refers to all cannabinoids, whether hemp-derived or not. Certain formulations may include THC at a ratio of 1:1 or 2:1 of total cannabinoids within the cannabinoid portion of the formulations, or THC may comprises any percentage of the total cannabinoid concentration of the formulation from 1 to 100%.

In one embodiment for topicals, the BPE may be in the range of 150-300 mg/ounce. In other embodiments, the range for BPE may be in the range of 200-250 mg/ounce. In yet other embodiments, the range may be 150-250 mg/ounce, 200-300 mg/ounce, or 50-150 mg/ounce.

In one embodiment for a topical, the BPE may be in one of the ranges set out above, plus at least 650 mg of total cannabinoids in 2.5 ounces of the topical. In one embodiment, the BPE may be in a range of 600-750 mg in 2.5 ounces of topical solution.

In one embodiment of a topical, the topical may contain at least 500 mg of cannabinoids for a 4 oz. solution. In another embodiment, the topical may contain at least 520 mg of cannabinoids for a 4 oz. solution. The 4 oz. solution may include BPE in a range of 700-1100 mg. In one embodiment, the BPE content is in the range of 800-1000 mg.

For the embodiments using THC, the cannabinoid content may be doubled or tripled, and the BPE range may remain the same. For example, if the solution has 520 mg total for the non-THC embodiment, and ratio of 1:1 THC to other cannabinoids, the total milligrams of cannabinoids, including THC, will be 1040 mg.

Consumables may have higher levels of BPE, although there is no limitation to the range of BPE in topicals. In one embodiment one dose of the consumable may contain at least 200 mg of BPE for pain/inflammation. Note that for consumables, the milligrams is milligrams per dose and the dose size may vary. The amount used depends upon many factors, so other embodiments may use less than 200 mg, a range of 100-500 mg, 100-400 mg, 200-500 mg, and 200-400 mg. Individual doses of BPE may comprises 25 mg, 50 mg, 100 mg, 200 mg, and 250 mg, as examples. The term “dose” as used here means one unit of consumable, such as one or two capsules, some number of drops of tincture, as examples and without limitation.

One example may comprise any number of milligrams of BPE with 5 mg of THC and at least 5 mg of other cannabinoids, to form a total cannabinoid dose of 10 mg, to form a pill or could put the contents into gel caps, pressed powder pills, or other consumables. Other doses of cannabinoids may be 5 mg, 10 mg, 25 mg, 50 mg, and 100 mg.

The combinations of the two compounds may include 5-100 mg cannabinoids, and 25-250 mg BPE. Any amount of BPE in this range, or in the range up to 400 mg, in any steps of 5 mg, combined with any amount of cannabinoids in the range of 5-100 mg per dose in any steps of 5 mg, is considered to be within the scope of the claims. For example, some formulation mays include 5 mg cannabinoids: 25 mg BPE; 10 mg of cannabinoids: 50 mg of BPE; 25 mg of cannabinoids: 100 mg BPE; 50 mg of cannabinoids: 200 mg of BPE; and 100 mg of cannabinoids: 250 mg of BPE. These are just examples to demonstrate some of the various combinations possible under the ranges of BPE and cannabinoids.

In addition to the overall cannabinoid content, the embodiments may include a main cannabinoid, such as CBD or CBG, plus a “minor profile” per gram. A “minor profile” includes those cannabinoids that are not the focus of a particular formulation. There is no limitation to any particular cannabinoids as either the main cannabinoid or the minor cannabinoids. Similarly, there is no limitation as to how many of the minor cannabinoids exist in the minor profile or their respective concentrations.

In one embodiment, the cannabinoid profile may contain a percentage per gram of the main cannabinoid plus 15-30% minor profile per gram. The minor profiles may include 4 or more other cannabinoids, or have as few as one other cannabinoid in the profile.

In one embodiment, a 2.5-ounce topical solution may have a total cannabinoid content (TCC) of at least 650 milligrams. One embodiment has 45-55% is the main cannabinoid, another embodiment has at least 40% of the main cannabinoid. Another embodiment has concentrations of minor cannabinoids of 0-7%, 1-3%, 0-3%, 1-5%, and 2-14%. Each percentage represents a range of minor cannabinoids. In one embodiment, the main cannabinoid is CBD and the minor cannabinoids are CBG, CBN, CBDV, CBDA, and CBC, possibly with or without other minors. In another embodiment, the minor cannabinoids may have a different sequence and different percentages of concentrations. The examples above are merely for ease of understanding and are not intended to limit the concentrations to any particular combination of minor cannabinoids in any percentages.

Another embodiment may comprise the 4 oz. solution mentioned above, having a TCC of at least 500 mg of cannabinoids, with 45-55% of the main cannabinoid, and minors in the range of 0-7%, 1-3%, 0-3%, 1-5% , and 2-14%, of the same minors above, when CBD is the main cannabinoid.

Another embodiment has the main cannabinoid at 15-30% of the TCC, with some variation of the minor profile per gram for a 4 oz. solution.

In any embodiment of the topicals, the TCC is at least 50%, in some embodiments it is 60% and in others, it is 70%, without limitation.

As mentioned previously, these formulations may also exist with THC added to them. THC can also be used as the primary, with CBD, CBG, etc., being part of the minor profile. In one embodiment, THC may comprise 100% of the cannabinoids in the formulation.

Although specific embodiments have been illustrated and described for purposes of illustration, it will be understood that various modifications may be made without departing from the spirit and scope of the attached claims.

What is claimed is:

1. A formulation comprising butterfly pea extract (BPE) and cannabinoids.

2. The formulation as claimed in claim 1, wherein the cannabinoids comprise one or more of cannabidiol, cannabidiolic acid, cannabinol, cannabigerol, cannabichromene, cannabidivarin, and THC.

3. The formulation as claimed in claim 1, wherein the cannabinoids are hemp-sourced cannabinoids.

4. The formulation as claimed in claim 1, wherein the cannabinoids contain THC at a ratio of at or between 1:1 and 2:1.

5. The formulation as claimed in claim 1, wherein the cannabinoids may have a profile of a major cannabinoid and one or more minor cannabinoids

6. The formulation as claimed in claim 1, wherein the formulation is a topical.

7. The formulation as claimed in claim 6, wherein the topical comprises a 4-ounce solution with a total cannabinoid content of at least 500 mg.

8. The formulation as claimed in claim 6, wherein the topical comprises a 4-ounce solution with a total cannabinoid content between 1 mg and 500 mg.

9. The formulation as claimed in claim 6, wherein the BPE has a concentration in one of the ranges of: 150-300 mg/ounce; 200-250 mg/ounce; 150-250 mg/ounce; 200-300 mg/ounce; and
50-150 mg/ounce.

10. The formulation as claimed in claim 6, wherein the cannabinoid has a concentration of at least 650 mg of total cannabinoids in 2.5 ounces of topical solution.

11. The formulation as claimed in claim 6, wherein the BPE has a concentration in the range of 600-750 mg in 2.5 ounces of topical solution.

12. The formulation as claimed in claim 6, wherein the cannabinoids has a concentration of one of either at least 500 mg of cannabinoids or at least 520 mg of cannabinoids in a 4 oz. solution.

13. The formulation as claimed in claim 12, wherein the BPE has a concentration in a range of either of 700-1100 mg or 800-1000 mg.

14. The formulation as claimed in claim 6, wherein the cannabinoid concentration includes a concentration of THC at a ratio of 1:1 to other cannabinoids.

15. The formulation as claimed in claim 13, wherein the cannabinoid concentration comprises 520 mg of THC and 520 mg of other cannabinoids.

16. The formulation as claimed in claim 1, wherein the formulation is a consumable.

17. The formulation as claimed in claim 16, wherein the consumable comprises one of tinctures, oils, water-soluble tablets, powder packets, capsules, pills, teas, powders, edibles, gummies, chocolates, and liquids.

18. The formulation as claimed in claim 16, wherein the BPE has a concentration of at least 200 mg.

19. The formulation as claimed in claim 16, wherein the BPE has a concentration in a range of one 100-500 mg, 100-400 mg, 200-500 mg, and 200-400 mg.

20. The formulation as claimed in claim 16, wherein the cannabinoids have a concentration in the range of 5-100 mg per dose.

21. The formulation as claimed in claim 16, wherein the BPE and cannabinoids have concentrations of one of 5 mg cannabinoids: 25 mg BPE; 10 mg of cannabinoids: 50 mg of BPE; 25 mg of cannabinoids: 100 mg BPE; 50 mg of cannabinoids: 200 mg of BPE; and 100 mg of cannabinoids: 250 mg of BPE.
